(19) United States
(12) Patent Application Publication
(10) Pub. No.: US 2024/0316320 A1
(43) Pub. Date: Sep. 26, 2024

(54) DILATABLE BILIARY DRAINS

(71) Applicants: Northwestern University, Evanston, IL (US); Northwestern Memorial HealthCare, Chicago, IL (US)

(72) Inventor: Ahsun Riaz, Chicago, IL (US)

(21) Appl. No.: 18/681,675

(22) PCT Filed: Aug. 16, 2022
(86) PCT No.: PCT/US2022/040437

§ 371 Date: Feb. 6, 2024

Related U.S. Application Data
(60) Provisional application No. 63/233,961, filed on Aug. 17, 2021.

(51) Int. Cl. A61M 27/00; A61M 29/02

(52) U.S. Cl. CPC A61M 27/002; A61M 29/02; A61M 2202/0403; A61M 2205/32; A61M 2207/00

(57) ABSTRACT

A biliary drain system includes a drain having a first end and a second end. The drain also includes a lumen that connects the first end to the second end. The system also includes a balloon mounted to the drain and configured to mount within a stricture of a bile duct of a patient. The balloon includes a first end, a second end, and a center portion. A size of the first end of the balloon is greater than a size of the center portion of the balloon.

### CROSS-REFERENCE TO RELATED APPLICATION

The present application claims the priority benefit of U.S. Provisional Patent App. No. 63/233,961 filed on Aug. 17, 2021, the entire disclosure of which is incorporated herein by reference.

### BACKGROUND

The bile ducts are a series of thin tubes that run from the liver to the small intestine. The primary purpose of the bile ducts is to allow a fluid (bile) to move from the liver and gallbladder into the small intestine, where the bile assists with the digestion of food. A blockage of the bile ducts due to biliary strictures or other causes can lead to obstructive jaundice, which is a condition that can prove fatal. Additionally, obstruction to the flow of bile through the bile ducts can lead to infections such as acute cholangitis, which can be catastrophic. Approximately one third of biliary strictures are due to benign etiologies. The etiologies of benign strictures can be broadly classified into lithiatic (biliary stone related) or non-lithiatic causes. The most common non-lithiatic etiology is post-surgical. Interventional Radiology (IR) can be used to place a drain in the biliary system to help manage biliary strictures and prevent damage caused by the stricture.

### SUMMARY

An illustrative biliary drain system includes a drain having a first end and a second end. The drain also includes a lumen that connects the first end to the second end. The system also includes a balloon mounted to the drain and configured to mount within a stricture of a bile duct of a patient. The balloon includes a first end, a second end, and a center portion. A size of the first end of the balloon is greater than a size of the center portion of the balloon.

In an illustrative embodiment, the drain includes a wire access area adjacent to the balloon, where the wire access area is configured to receive a wire to puncture the balloon. In one embodiment, the wire access area is a through hole. In another embodiment, the wire access area is a weakened area of a wall of the drain. In another embodiment, a main portion of the drain is made of a first material and the weakened area of the wall of the drain is made of a second material to facilitate a puncture through the wire access area. The system can also include the wire, where the lumen of the drain is configured to receive the wire. In another illustrative embodiment, the drain includes a radio-opaque border that surrounds at least a portion of the wire access area. One or more radio-opaque markers can form the radio-opaque border in one embodiment.

In another embodiment, the drain includes a balloon valve lumen that is configured to receive a gas to inflate the balloon. The drain can also include a first plurality of drain holes positioned on a first portion of the drain that is between the first end of the drain and the balloon. The drain can further include a second plurality of drain holes positioned on a second portion of the drain that is between the second end of the drain and the balloon. In an illustrative embodiment, a size of the second end of the balloon is greater than the size of the center portion of the balloon. The balloon can be a hybrid balloon that is a hybrid between a high pressure balloon and a compliant balloon.

An illustrative method of forming a biliary drain includes forming a drain having a first end and a second end, where the drain is formed to include a lumen that connects the first end to the second end. The method also includes forming a balloon that has a first end, a second end, and a center portion, where the balloon is formed such that a size of the first end of the balloon is greater than a size of the center portion of the balloon upon inflation of the balloon. The method can further include mounting the balloon to the drain.

In an illustrative embodiment, forming the drain includes forming a wire access area adjacent to a position at which the balloon is mounted to the drain, where the wire access area is configured to receive a wire to puncture the balloon. The wire access area can be formed as a through hole. The wire access area can also be formed as a weakened area of a wall of the drain such that the weakened area is more susceptible to a puncture than a remainder of the wall of the drain. A radio-opaque border is also formed to surround at least a portion of the wire access area. In one embodiment, forming the drain includes forming a balloon valve lumen in the drain that is configured to receive a gas to inflate the balloon. Forming the drain also includes forming a first plurality of drain holes positioned on a first portion of the drain that is between the first end of the drain and the balloon, and forming a second plurality of drain holes positioned on a second portion of the drain that is between the second end of the drain and the balloon.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 is a diagrammatic representation of a traditional transhepatic internal/external biliary drain in accordance with an illustrative embodiment.

FIG. 2A is a pre-angioplasty cholangiographic image demonstrating a severe anastomotic biliary stricture with a wire passing from the skin, through the liver, bile ducts, anastomotic stricture, and into the bowel in accordance with an illustrative embodiment.

FIG. 2B depicts a cholangioplasty using a conventional 8 mm balloon with the wire across the stricture in accordance with an illustrative embodiment.

FIG. 2C is a post-angioplasty cholangiographic image demonstrating mildly improved flow through the anastomotic biliary stricture with a wire across the stricture in accordance with an illustrative embodiment.

FIG. 2D depicts an internal external biliary drain placed across the stricture in accordance with an illustrative embodiment.

FIG. 3 depicts a diagrammatic form of the proposed biliary stricture treatment system in accordance with an illustrative embodiment.

FIG. 4 depicts a diagrammatic form of the proposed biliary stricture treatment system in accordance with another illustrative embodiment.

FIG. 5A depicts an external appearance of a portion of a drain in accordance with a first illustrative embodiment.

FIG. 5B depicts an external appearance of a portion of a drain in accordance with a second illustrative embodiment.

FIG. 6 is a cross-sectional view of a balloon/drain lumen that demonstrates the ability to puncture the balloon with an appropriately positioned wire in accordance with an illustrative embodiment.

FIG. 7 depicts a variation of the dilatable biliary drain that can be placed through the jejunum of a patient in accordance with an illustrative embodiment.

### DETAILED DESCRIPTION

The biliary system drains important by-products of liver metabolism, and is integral to life. Biliary strictures, which refers to narrowing or blockage of biliary tubes, are difficult to address using minimally invasive methods. Traditional treatment methods for biliary strictures include the use of percutaneous biliary drains. However, traditional percutaneous biliary drains are constant reminders of the patient's health issues, and are also associated with pain, skin irritation, and leakage. Sleeping and performing routine tasks can also be very uncomfortable for patients using a traditional biliary drain. Additionally, most biliary drains are approximately 3 millimeters (mm) in diameter while the normal bile duct is approximately 6 mm in diameter. Use of a 6 mm diameter drain would lead to an increased amount of pain and discomfort for the patient. As a result, smaller diameter drains are used, which reduces overall drain efficiency due to the space between the drain and the bile duct.

As an example, the inventors retrospectively studied all biliary drains placed in the last ten years at a given medical institution. The total number of drains was 463. The total number of exchanges was 1182 with a mean number of 3.94 (range: 1-32). The mean duration was 79 days (range 1-2402 days). These numbers demonstrate that the current biliary stricture management protocols are limited. The drains have a significant impact on the quality of life and are associated with complications. This also has an impact on costs associated with multiple hospital visits and procedures. As discussed in more detail below, described herein is a dilatable biliary drain that can improve the quality of life, decrease complications associated with prolonged drainage, and potentially decrease costs associated with prolonged drainage and exchanges.

Patients suffering from biliary strictures can be asymptomatic, and in some situations the stricture(s) can be found incidentally during routine imaging performed due to other causes. Alternatively, patients can be symptomatic and present with pain, jaundice, fatigue, nausea, and vomiting. Fever can also be present if there is infection (cholangitis). With respect to diagnosis, benign biliary strictures can be diagnosed using laboratory tests to determine serum bilirubin levels and alkaline phosphatase levels. An elevated leukocyte count suggests infection. Additionally, serum tumor markers should be evaluated to exclude malignancy. Imaging evaluation using computed tomography (CT) or magnetic resonance cholangiopancreaticography (MRCP) can confirm upstream biliary ductal dilatation and the location of the stricture while excluding malignant etiologies.

Peroral endoscopic interventions by gastroenterology remain the first line treatment modality to manage benign strictures. Endoscopic retrograde cholangiopancreaticography (ERCP) may fail in approximately 10-15% cases which leads to intervention by interventional radiology (IR) specialists. There are instances where IR specialists intervene before gastrointestinal (GI) specialists, such as in patients who have had prior surgery making it difficult for GI specialists to advance the endoscope to the biliary-enteric anastomosis. Interventional radiology has also been the mainstay for treating strictures in the pediatric population due to the inability to advance large scopes in infants and toddlers. However, it is noted that IR can perform cholangioscopy, biopsy, and lithotripsy using endoscopes that are less than 4 mm (12 French) in diameter. This not only helps with management of the stricture, but also helps with addressing the etiology of the stricture.

As discussed, biliary drains are routinely placed using Interventional Radiology to help treat biliary strictures. Approximately one third of these routines are for benign strictures. The biliary drain helps to drain bile internally into the bowel and/or externally into a drainage bag, which allows for relief from obstructive jaundice and preserves liver function. Described below are several techniques that are currently used to treat biliary strictures.

One technique to help treat biliary strictures is the use of a prolonged biliary drain. With this technique, physicians have used a structured approach to managing benign biliary structures, by sequentially upsizing the drains and then leaving the drain in the patient for up to 6 months. This technique has resulted in primary patency rates of 81% for orthotopic liver transplant (OLT) patients (mean follow-up, 20 months) and 90% and 100%, respectively for non-OLT patients (mean follow-up, 13 months). However, prolonged placement of large drains (especially in the pediatric population) is very lifestyle limiting, and can cause significant pain to the patient.

Another technique for treating biliary strictures is the use of dual catheters. Large bore catheters can be avoided using such a dual catheter technique, in which a drain is placed through one of the side holes of an existing biliary drain. Alternatively, instead of a secondary catheter, a balloon catheter can be placed through an existing pigtail catheter and advanced through one of the side holes across the target stricture site. This technique has been shown to have a high primary technical and clinical success rate, without stricture recurrence. However, leakage at the site of the additional drain/balloon catheter exiting the existing drain can occur, which puts the patient at risk.

Another technique to treat biliary strictures is a cholangioplasty, in which conventional or cutting balloons are used in an effort to help dilate the stricture. The normal common bile duct measures approximately 6-8 mm (18-24 French). Drains of this size would be extremely uncomfortable for patients. Hence, when patients come back for routine exchange of the biliary drain, it is routine practice to place a conventional or cutting balloon to perform cholangioplasty of the strictured segment. However, the stricture is only dilated while the balloon is being inflated in the IR suite and the patient has to go back home with only a 2.7-4.7 millimeter (mm) diameter drain, which does not allow the stricture to expand further until the patient comes back for another IR procedure.

Yet another technique to treat biliary strictures is stent placement. Metal or plastic stents can be placed via a percutaneous approach if the patient is able to have the stent replaced/removed by an endoscopic route. A limitation of this technique is that stents occlude. As a result, in some situations, the biliary drain has to be replaced, which re-exposes the patient to the risks of drain placement such as liver injury and bleeding.

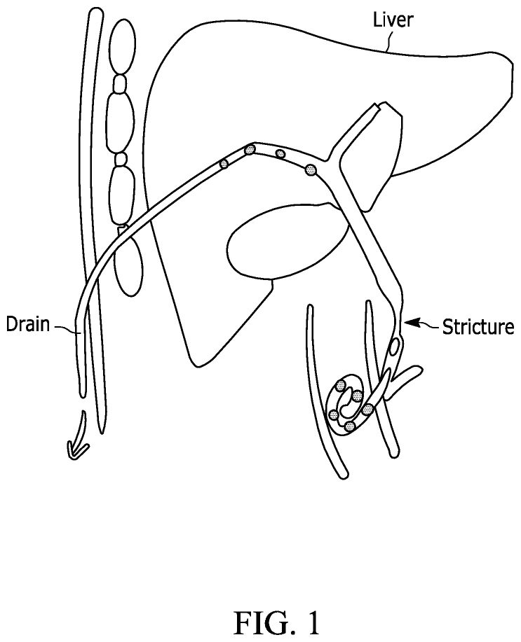

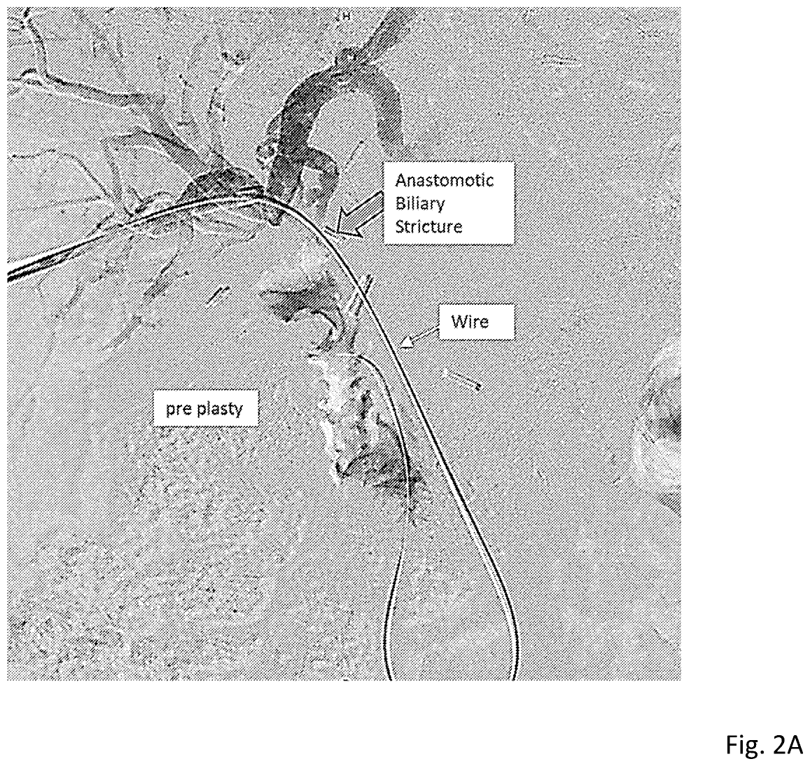

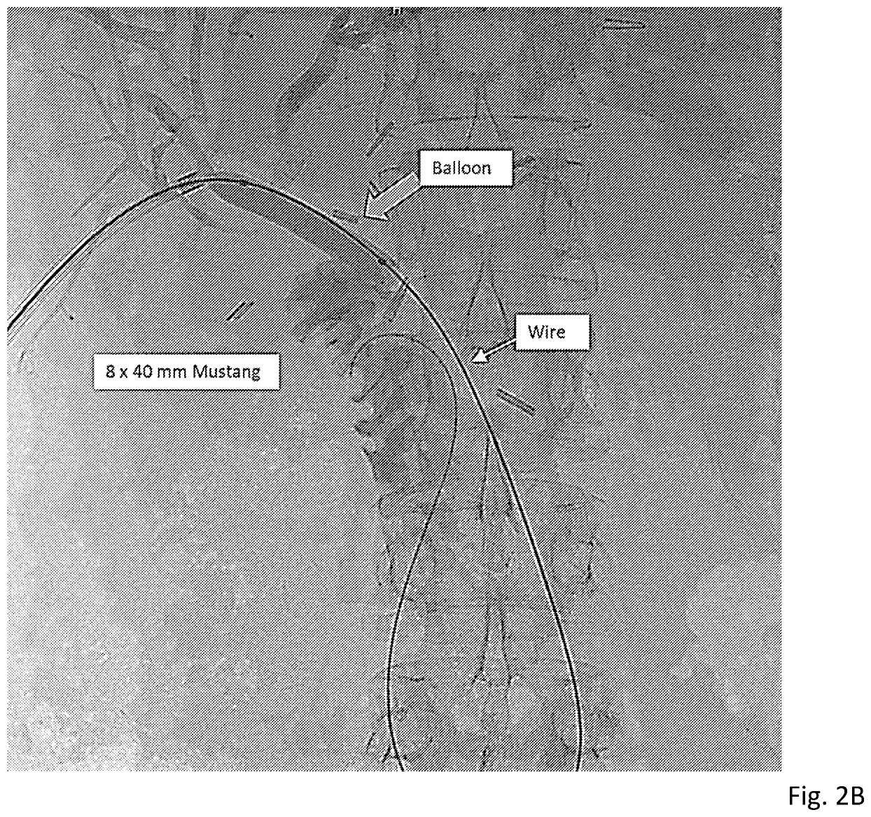

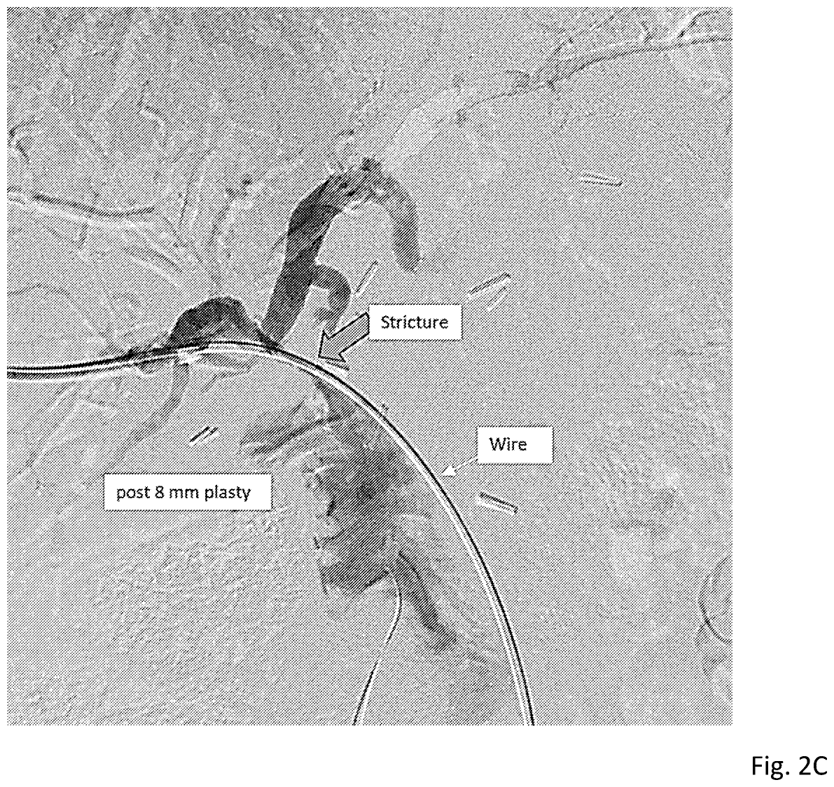

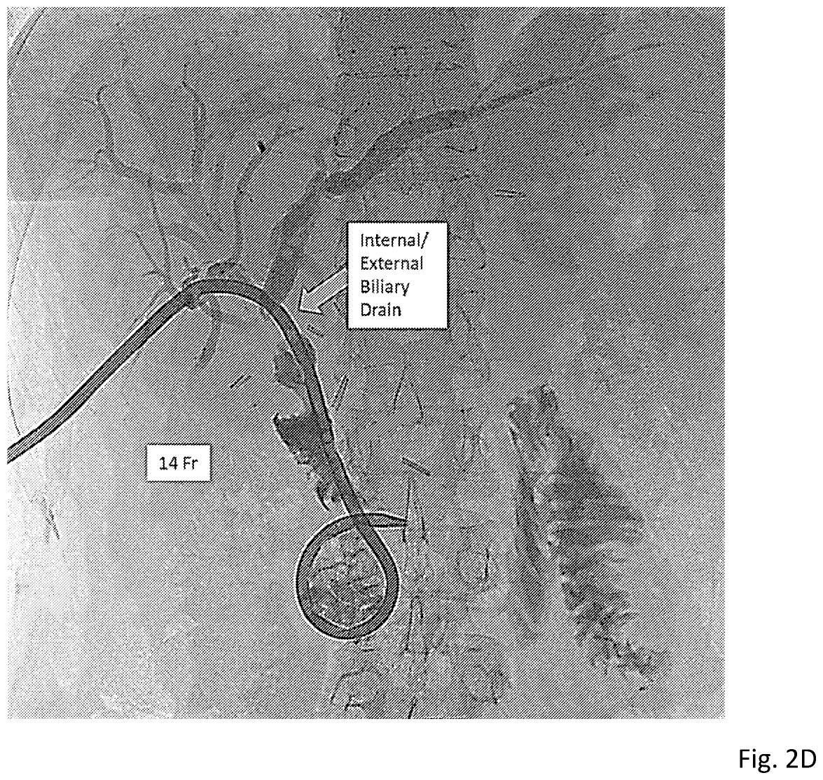

FIG. 1 is a diagrammatic representation of a traditional transhepatic internal/external biliary drain in accordance with an illustrative embodiment. Using a percutaneous transhepatic or transjejunal approach, 2.7-4.7 mm (8-14 French) drains can be placed across the stricture. FIGS. 2A-2D are images from a 50 year old female who underwent a percutaneous transhepatic cholangiogram for a post-surgical biliary stricture. As shown in the figures, interventional radiology placed a drain. Prior to placement of a drain, cholangioplasty of the stenosis was performed. This patient had the drain for more than 6 months.

Specifically, FIG. 2A is a pre-angioplasty cholangiographic image demonstrating a severe anastomotic biliary stricture with a wire passing from the skin, through the liver, bile ducts, anastomotic stricture, and into the bowel in accordance with an illustrative embodiment. FIG. 2B depicts a cholangioplasty using a conventional 8 mm balloon with the wire across the stricture in accordance with an illustrative embodiment. FIG. 2C is a post-angioplasty cholangiographic image demonstrating mildly improved flow through the anastomotic biliary stricture with a wire across the stricture in accordance with an illustrative embodiment. It is noted that the bile duct did not maintain the diameter of the balloon due to the elastic nature of the stricture. FIG. 2D depicts an internal external biliary drain placed across the stricture in accordance with an illustrative embodiment.

Described herein are methods and systems to help patients with obstructive biliary disease and other biliary drainage issues. The methods described herein treat benign biliary strictures by dilating the strictures to at least normal bile duct diameter for a prolonged time period, and this is done without having a drain of that size exiting the patient. The proposed techniques can allow for decreased time with an installed drain and increased luminal gain of the stricture. More specifically, described herein is a biliary drain system with a specially designed balloon dilatable segment. The system allows for prolonged and sustained dilatation of the stricture to an acceptable lumen, without having a large external drain. In an illustrative embodiment, the system will allow for dilatation of the stenosis beyond the diameter of the drain (usually 10-14 French). The increased resultant diameter of the stricture will allow for early drain removal and hence improve quality of life for the patient.

The proposed system includes an internal-external biliary drain with a balloon mounted on a segment of the drain that crosses the stenosis. In one embodiment, the balloon can be a high pressure non-compliant balloon. In another embodiment, to help prevent pressure necrosis of the bile duct, the balloon can be a hybrid balloon that is between a high pressure non-compliant balloon and a compliant balloon. As an example, compliant (i.e., elastometric) balloons can be made of silicone or polyurethane. Non-compliant balloons (i.e., high pressure balloons) can be made of made of materials such as co-extrusions of nylon and Pebax (NyBax®; Boston Scientific; Marlborough; MA). In an illustrative embodiment, the proposed hybrid balloon can be a combination of compliant balloon material(s) at the proximal and distal ends and non-compliant balloon material(s) in the mid-balloon between the proximal and distal ends.

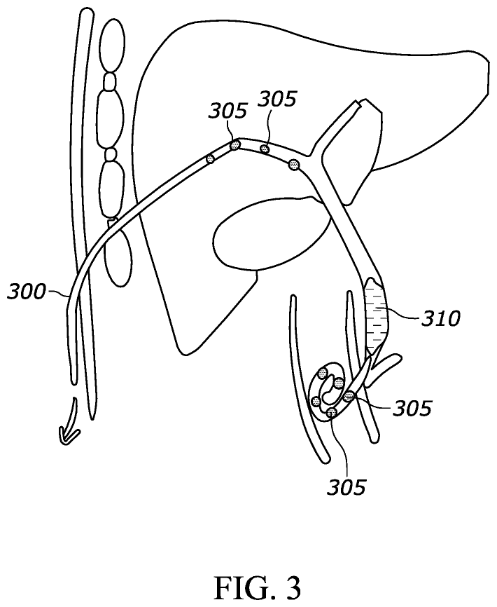

In an illustrative embodiment, the balloon is circumferentially mounted onto the drain using, for example, an adhesive. In such an embodiment, the balloon is unable to slide/move along the drain. The balloon is formed to include a lumen (e.g., shaped and sized to receive the drain) that surrounds a portion of the drain that is to be positioned through the stricture. FIG. 3 depicts a diagrammatic form of the proposed biliary stricture treatment system in accordance with an illustrative embodiment. As shown, the drain 300 includes drain (side) holes 305. The drain holes 305 are positioned above and below a balloon 310 that will allow bile to flow across the stricture through the lumen of the drain. It is noted that a balloon with a uniform diameter, such as the balloon 310 shown in FIG. 3, may be prone to migration.

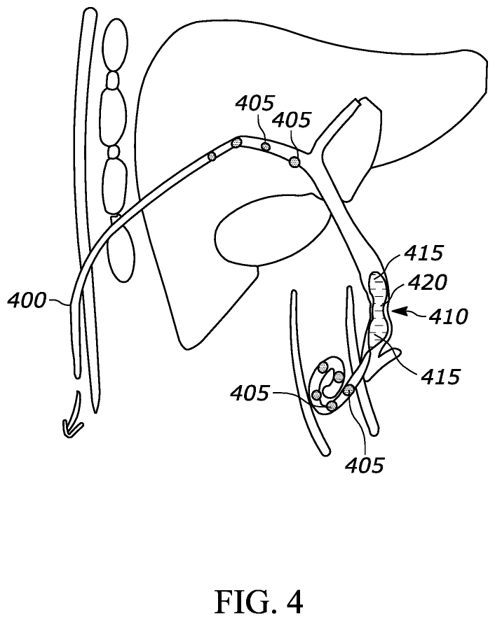

FIG. 4 depicts a diagrammatic form of the proposed biliary stricture treatment system in accordance with another illustrative embodiment. In FIG. 4, the drain 400 includes drain holes 405 positioned above and below of a balloon 410. The balloon 410 can be a high-pressure balloon or a hybrid balloon as discussed herein, and is integral to the function of the proposed system. The balloon 410 has dilated ends 415 (i.e., a first dilated end and a second dilated end) that are slightly more dilated than a middle portion 420 to limit migration (i.e., watermelon seeding) of the balloon 410 from the stricture. As discussed herein, the proposed dilatable biliary drain can be used to perform a persistent dilatation of the stricture for a course of days to weeks to help the stricture mold to the diameter of the balloon and provide prolonged resolution of the benign biliary stricture.

More specifically, when dilated, the balloon 410 is generally cylindrical in shape, but has areas of varying diameter. The first end of the balloon 410 and the second end of the balloon 410 each have a diameter (or size) which is greater than the diameter (or size) of the middle (or center) portion 420 of the balloon. As an example, the middle portion 420 of the balloon 410 may have a diameter of 6 mm and the ends of the balloon 410 can have diameters of 8 mm. Alternatively, different sizes may be used. The first and second ends of the balloon 410 can be of the same size or can have different sizes, depending on the specific shape and configuration of the stricture. This shape (i.e., barbell shape) of the balloon 410 allows the stricture to stabilize the balloon to prevent movement thereof. The side drain holes 405 above and below the balloon 410 allow bile to flow across the stricture through the lumen of the drain 400.

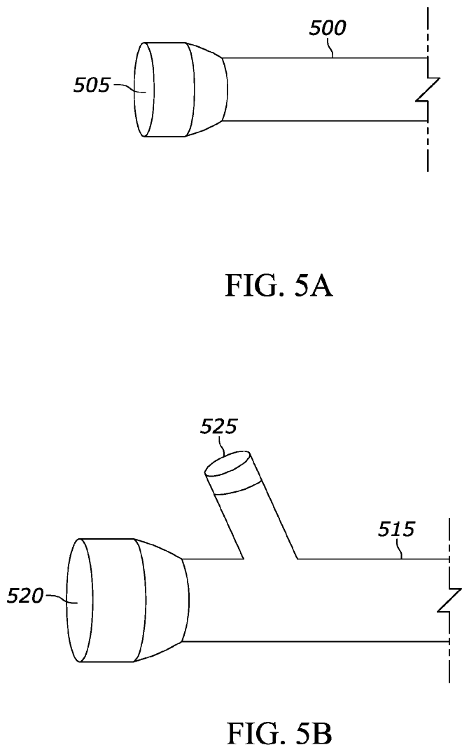

FIG. 5A depicts an external appearance of a portion of a drain 500 in accordance with a first illustrative embodiment. FIG. 5B depicts an external appearance of a portion of a drain 515 in accordance with a second illustrative embodiment. The drain 500 includes a lumen 505 which is connected to a bag, and the drain 515 similarly includes a lumen 520. The drain 515 of FIG. 5B also includes a balloon valve lumen 525 that is used to deflate/inflate the balloon. In such an embodiment, there are two lumens (e.g., side by side) in the dilatable biliary drain. The main lumen 520 is for the flow of bile, and the second lumen (i.e., the balloon valve lumen 525) can be attached to the balloon valve to allow for inflation/deflation of the balloon. The balloon can be inflated with any gas/fluid that is safe for the patient.

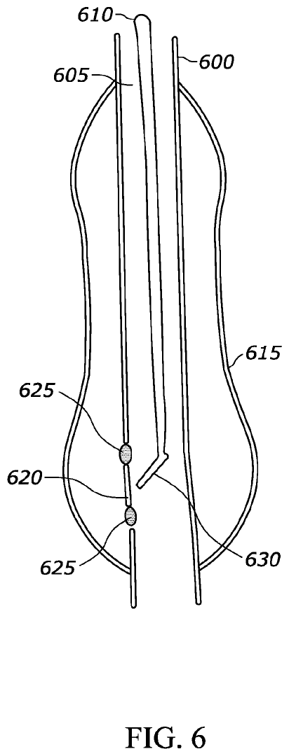

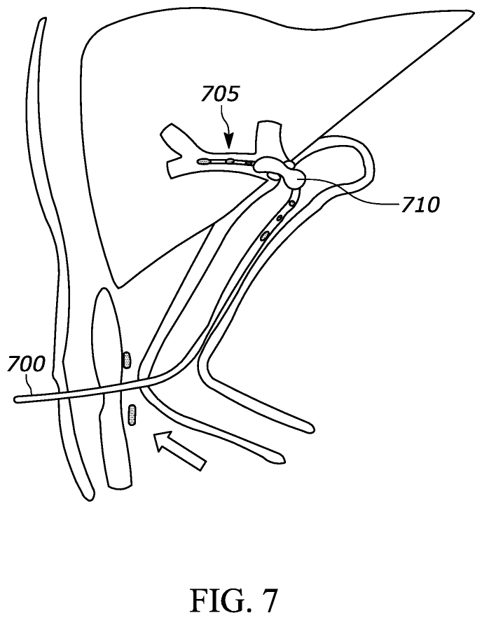

In an illustrative embodiment, the balloon should be able to deflate in more than 90% of cases using an external port, such as the balloon valve lumen 525 depicted in FIG. 5B. However, in cases where it is not possible to deflate the balloon using regular aspiration techniques from the balloon valve lumen 525, a safety mechanism incorporated into the system can be used to puncture the balloon to allow for deflation. In one embodiment, the safety mechanism includes a wire that can be manipulated through the drain to puncture the balloon via the lumen of the drain in a targeted fashion. This is presented in diagrammatic form in FIG. 6. Specifically, FIG. 6 is a cross-sectional view of a balloon/drain lumen that demonstrates the ability to puncture the balloon with an appropriately positioned wire in accordance with an illustrative embodiment.

As shown, a drain 600 includes a lumen 605 into which a small wire 610 can be placed to puncture a balloon 615 that is mounted to the drain 600. The drain 600 also includes a wire access area 620 that allows the wire 610 to access the balloon 615. The wire access area 620 is at least partially surrounded by a radio-opaque border 625 that can be used by a physician to locate the wire access area 620 via imaging. In an illustrative embodiment, the wire 610 can be placed through the wire access area 620 in the drain 605 and into the adjacent balloon 615 to puncture the balloon 615 for deflation. As shown, the wire 610 includes a bent wire tip 630 that enters the wire access area 620 to perform the puncturing. The bent wire tip 630 can be at a 45 degree angle relative to a main body of the wire 610. Alternatively, a different angle may be used.

In an illustrative embodiment, the radio-opaque border 625 can be formed by one or more radio-opaque markers that are adjacent to the wire access area 620 and that are used to mark the wire access area 620. In one embodiment, the wire access area 620 can be a small through-hole in the drain 600. Alternatively, the wire access area 620 can be a weakened area of the drain 600 that is able to be penetrated by the wire. In such an embodiment, the weakened area of the drain that forms the wire access area can be made from the same material as the drain, but can be significantly thinner than the rest of the drain. For example, the weakened area that forms the wire access area 620 can have a thickness that is 50% of the thickness of the rest of the drain. Alternatively, the weakened area can have a thickness that is 10%, 20%, 30%, 40%, etc. of the thickness of the rest of the drain, depending on the type of materials used. In another alternative embodiment, the drain may be made from a first material, and the weakened area can be formed from a second material that is easier to penetrate with a wire than the first material.

The proposed system can also be used to perform percutaneous transjejunal biliary interventions. FIG. 7 depicts a variation of a dilatable biliary drain 700 that can be placed through the jejunum of a patient in accordance with an illustrative embodiment. It is known that the internal pigtail (shown in FIG. 1) of routinely placed IR drains try to coil in the bile ducts and can lead to bile duct injury due to pressure from the tip trying to coil inside of a small duct branch. To prevent this from happening, an internal tip 705 of the drain 700 shown in FIG. 7 is straight. The drain 700 can be kept in place by a balloon 710 which has larger diameter ends than its middle. The use of the balloon 710 obviates the need for the internal pigtail that is used in traditional systems. The wide arrow at the bottom of FIG. 7 points towards a modified Hutson loop, which is a route of percutaneous entry into the bowel to perform procedures in the biliary system.

In an illustrative embodiment, the proposed design can be used for treating anastomotic strictures with a segment of duct (without branching above the stricture), as shown in FIG. 1. In patients with hilar strictures (at the branch point of the duct), multiple dilatable biliary drains can be placed as balloon inflation may occlude flow from the contralateral side. This is seen in FIG. 7, where the flow from the left biliary system could potentially be occluded by the right sided dilatable biliary drain.

As discussed throughout, the proposed system can be used for the management of benign biliary strictures using the percutaneous transhepatic approach. The system can also be used for management of benign biliary strictures using a percutaneous transjejunal approach. The proposed system can similarly be used for benign ureteric strictures as well.

As also discussed above, current technologies rely on percutaneous biliary drainage (drain going through the skin, liver, into the bile ducts, through the bile ducts, and into the bowel). Interventional radiology drains are long uniform diameter tubes as they are placed externally. The presence of a balloon along a segment of the drain allows for very prolonged dilatation of the stricture leading to early removal of drain and increased luminal gain.

The proposed drain is an innovative solution that can be incorporated into the management of numerous patients with benign biliary strictures. Currently, drains just serve the purpose of drainage in between cases. With the proposed dilatable biliary drain option, they will be able to dilate and treat while the drain is in position. This design of the dilatable biliary drain will improve patient care and quality of life. As discussed, benign biliary strictures have traditionally required prolonged multi-step treatment, and the proposed methods and system can decrease the number of procedures needed and the overall duration of treatment.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more”.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A biliary drain system comprising:
a drain having a first end and a second end, wherein the drain also includes a lumen that connects the first end to the second end; and
a balloon mounted to the drain and configured to mount within a stricture of a bile duct of a patient, wherein the balloon includes a first end, a second end, and a center portion, and wherein a size of the first end of the balloon is greater than a size of the center portion of the balloon.

2. The system of claim 1, wherein the drain includes a wire access area adjacent to the balloon, wherein the wire access area is configured to receive a wire to puncture the balloon.

3. The system of claim 2, wherein the wire access area comprises a through hole.

4. The system of claim 2, wherein the wire access area comprises a weakened area of a wall of the drain.

5. The system of claim 4, wherein the drain is made of a first material and the weakened area of the wall of the drain is made of a second material to facilitate a puncture through the wire access area.

6. The system of claim 2, further comprising the wire, wherein the lumen of the drain is configured to receive the wire.

7. The system of claim 2, further comprising a radio-opaque border that surrounds at least a portion of the wire access area.

8. The system of claim 7, further comprising one or more radio-opaque markers that form the radio-opaque border.

9. The system of claim 1, wherein the drain includes a balloon valve lumen that is configured to receive a gas to inflate the balloon.

10. The system of claim 1, wherein the drain includes a first plurality of drain holes positioned on a first portion of the drain that is between the first end of the drain and the balloon.

11. The system of claim 10, wherein the drain includes a second plurality of drain holes positioned on a second portion of the drain that is between the second end of the drain and the balloon.

12. The system of claim 1, wherein a size of the second end of the balloon is greater than the size of the center portion of the balloon.

13. The system of claim 1, wherein the balloon comprises a hybrid balloon that is a hybrid between a high pressure balloon and a compliant balloon.

14. A method of forming a biliary drain, the method comprising:
forming a drain having a first end and a second end, wherein the drain is formed to include a lumen that connects the first end to the second end;
forming a balloon that has a first end, a second end, and a center portion, wherein the balloon is formed such that a size of the first end of the balloon is greater than a size of the center portion of the balloon; and
mounting the balloon to the drain.

15. The method of claim 14, wherein forming the drain includes forming a wire access area adjacent to a position at which the balloon is mounted to the drain, wherein the wire access area is configured to receive a wire to puncture the balloon.

16. The method of claim 15, wherein the wire access area is formed as a through hole.

17. The method of claim 15, wherein the wire access area is formed as a weakened area of a wall of the drain such that the weakened area is more susceptible to a puncture than a remainder of the wall of the drain.

18. The method of claim 15, further comprising forming a radio-opaque border that surrounds at least a portion of the wire access area.

19. The method of claim 14, wherein forming the drain includes forming a balloon valve lumen in the drain that is configured to receive a gas to inflate the balloon.

20. The method of claim 14, wherein forming the drain includes forming a first plurality of drain holes positioned on a first portion of the drain that is between the first end of the drain and the balloon, and forming a second plurality of drain holes positioned on a second portion of the drain that is between the second end of the drain and the balloon.
